(19) United States
(12) Patent Application Publication
(10) Pub. No.: US 2011/0152932 A1
(43) Pub. Date: Jun. 23, 2011

(54) Flexible Spine Fixing Structure

(75) Inventors: Jaw-Lin Wang, Taipei City (TW); Dar-Ming Lai, Taipei City (TW); Shan-Chang Chueh, Taipei City (TW); Chih-Ming Wu, Hsinchu City (TW); Pei-Yu Peng, Toufen Township (TW)

(73) Assignee: INDUSTRIAL TECHNOLOGY RESEARCH INSTITUTE, Hsinchu (TW)

(21) Appl. No.: 12/860,085

(22) Filed: Aug. 20, 2010

(30) Foreign Application Priority Data
Dec. 21, 2009 (TW) 98143986

(51) Int. Cl. A61B 17/70

(52) U.S. Cl. 606/246

(57) ABSTRACT

A flexible spine fixing structure is provided. The flexible spine fixing structure is used for fixing adjacent two or more vertebras. The flexible spine fixing structure comprises a first flexible part, a first fixing part and a second fixing part. The first fixing part and the second fixing part are respectively connected to two ends of the first flexible part for fixing in the first vertebra. The second flexible part is directly connected to the first flexible part. The third fixing part and the fourth fixing part are respectively connected to two ends of the second flexible part for fixing in the second vertebra.

This application claims the benefit of Taiwan application Serial No. 98143986, filed Dec. 21, 2009, the subject matter of which is incorporated herein by reference.

### BACKGROUND

1. Technological Field

The disclosure relates in general to a flexible spine fixing structure, and more particularly to a flexible spine fixing structure for fixing in two or more vertebras.

2. Description of the Related Art

There are many ways for fixing spine vertebra. In general, the vertebras are fixed by multiple horizontal fixing plates, which are further bonded by a vertical hard structure.

Alternatively, another fixing structure is formed by two plates and a number of nails. A hole is formed on the plate for fixing in the angle, and the nails pass through two vertebras at an angle of 20-60 degrees for fixing in the two vertebras.

No matter what fixing method is employed, the spine being fixed is deprived of activities, and the surgery recipient can not move his/her spine such as forward or backward at fixed levels. Besides, the vertebras having been fixed and deprived of activities for a long time will gradually be fused into one single vertebra, and the vertebras being fixed and their adjacent vertebras will degenerate fast and loose activities for good.

### SUMMARY

The disclosure is directed to a flexible spine fixing structure. The flexibility of the flexible spine fixing structure may enables the fixed vertebras to move, and provides the patients with higher degree of activities, so that the patient's discomfort after surgery is reduced, and spine degeneration will be avoided.

According to a first aspect of the present disclosure, a flexible spine fixing structure is provided. The flexible spine fixing structure is for fixing in a first vertebra and a second vertebra of the spine. The flexible spine fixing structure comprises a first flexible part, a first fixing part, a second fixing part, a second flexible part, a third fixing part and a fourth fixing part. The first fixing part and a second fixing part are connected to two ends of the first flexible part respectively for fixing in the first vertebra. The second flexible part is directly connected to the first flexible part. The third fixing part and the fourth fixing part are connected to two ends of the second flexible part respectively for fixing in the second vertebra.

The disclosure will become apparent from the following detailed description of the preferred but non-limiting embodiments. The following description is made with reference to the accompanying drawings.

### BRIEF DESCRIPTION OF THE DRAWINGS

FIG. 1 (not illustrated the fastening element) shows a schematic view of a flexible spine fixing structure disposed in a vertebra according to a first embodiment of the disclosure;

FIG. 2 shows a schematic view of the flexible spine fixing structure of FIG. 1;

FIG. 3 shows a schematic view of a flexible spine fixing structure according to a second embodiment of the disclosure;

FIG. 4 shows a schematic view of a flexible spine fixing structure according to a third embodiment of the disclosure;

FIG. 5 shows a schematic view of a flexible spine fixing structure according to a fourth embodiment of the disclosure;

FIG. 6 shows a schematic view of a flexible spine fixing structure according to a fifth embodiment of the disclosure;

FIG. 7 shows a schematic view of a flexible spine fixing structure according to a sixth embodiment of the disclosure; and

FIG. 8 shows a schematic view of a flexible spine fixing structure according to a seventh embodiment of the disclosure.

### DETAILED DESCRIPTION

### First Embodiment

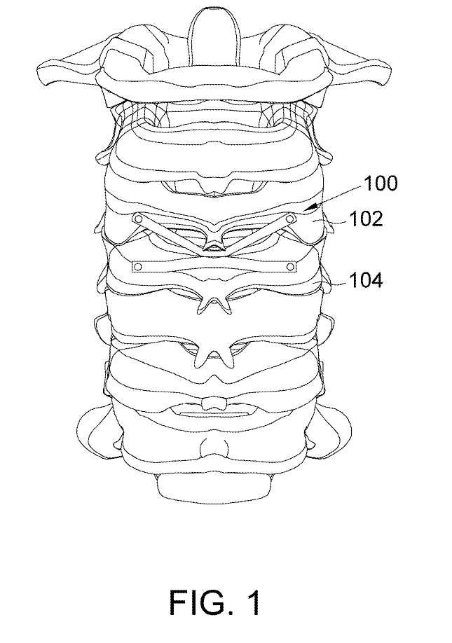

Referring to FIG. 1 (the fastening element is not illustrated), a schematic view of a flexible spine fixing structure disposed in a vertebra according to a first embodiment of the disclosure is shown. The flexible spine fixing structure 100 is for fixing in two vertebras such as the first vertebra 102 and the second vertebra 104 which are adjacent to each other. The first vertebra 102 and the second vertebra 104 are not limited to any specific vertebra, and may be any two vertebras of the cervical vertebra, the thoracic vertebra or the lumbar vertebra of the spine.

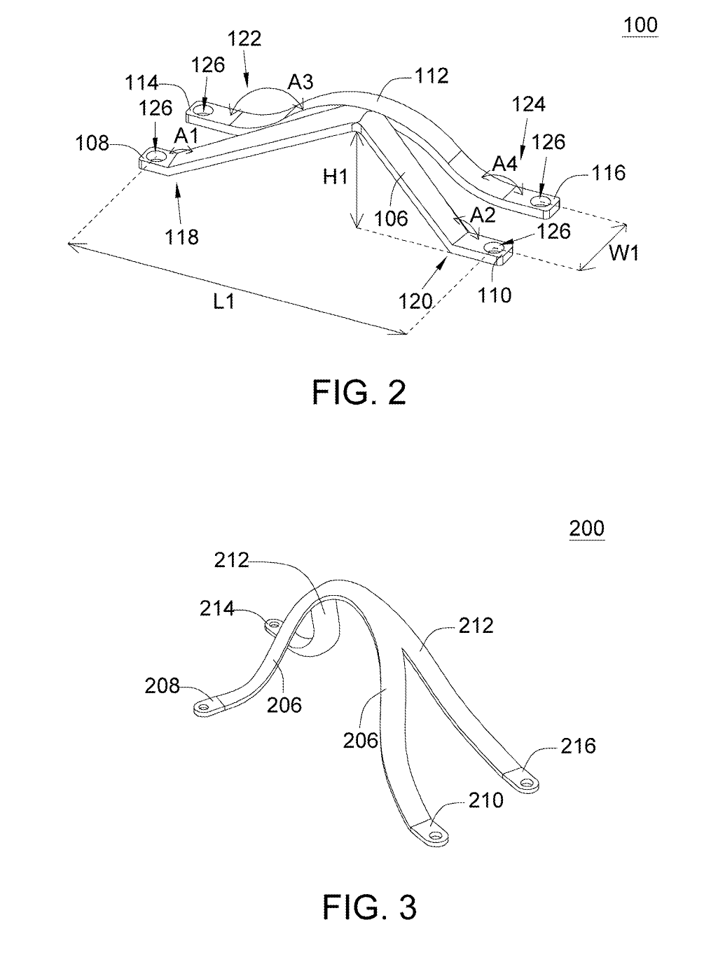

Referring to FIG. 2, a schematic view of the flexible spine fixing structure of FIG. 1 is shown. The flexible spine fixing structure 100 comprises a first flexible part 106, a first fixing part 108, a second fixing part 110, a second flexible part 112, a third fixing part 114 and a fourth fixing part 116. Any of the first fixing part 108, the second fixing part 110, the third fixing part 114 and the fourth fixing part 116 has a through hole 126, which allows a number of fastening elements such as screws (not illustrated) to pass through and fasten the flexible spine fixing structure 100 in the first vertebra 102 and the second vertebra 104.

The second flexible part 112 is directly connected to the first flexible part 106, so that the entire flexible spine fixing structure 100 is flexible. That is, after the flexible spine fixing structure 100 is fixed on the vertebra, the vertebra being fixed is still moveable and allows the patient to move forward, backward, leftward or rightward, and provides the patient with higher degree of activities, so that the patient's discomfort after surgery is reduced, and spine degeneration will be avoided.

The first fixing part 108 and the second fixing part 110 are connected to the first end 118 and the second end 120 of the first flexible part 106 respectively for fixing in the first vertebra 102. The third fixing part 114 and the fourth fixing part 116 are connected to the third end 122 and the fourth end 124 of the second flexible part 112 respectively for fixing in the second vertebra 104.

The first flexible part 106 and the second flexible part 112 of the present embodiment of the disclosure can achieve flexibility through the design or selection of the shape, the material, the cross-section or other parameters. The details of the structures of the first flexible part 106 and the second flexible part 112 are disclosed below.

The first fixing part 108 and the second fixing part 110 intersect the first flexible part 106. The third fixing part 114 and the fourth fixing part 116 intersect the second flexible part 112. That is, an angle is contained between the first fixing part 108 and the first flexible part 106, an angle is contained between the second fixing part 110 and the first flexible part 106, an angle is contained between the third fixing part 114 and the second flexible part 112, and an angle is contained between the fourth fixing part 116 and the second flexible part 112.

The first flexible part 106 may be straight, and the second flexible part 112 may be arched. An angle A1 is contained between the first fixing part 108 and the first flexible part 106. An angle A2 is contained between the second fixing part 110 and the first flexible part 106. An angle A3 is contained between the third fixing part 114 and the second flexible part 112. An angle A4 is contained between the fourth fixing part 116 and the second flexible part 112. The contained angles are obtuse angles, preferably but not limited to be ranging between 120-150 degrees, so that the first flexible part 106 and the second flexible part 112 have flexibility.

Besides, the cross-sections of the first flexible part 106 and the second flexible part 112 may be rectangular, circular or elliptical, and the first flexible part 106 and the second flexible part 112 may be flat or slim and long, so that the first flexible part 106 and the second flexible part 112 may generate suitable deformation when receiving a force.

The first flexible part 106 and the second flexible part 112 may be made from metal, polymer, flexible materials, elastic materials or combination thereof. For example, the metal may be realized by bio-compatible metal, stainless steel, memory alloy or a metal containing titanium such as pure titanium or titanium alloy. The polymer may be realized by polyether ether ketone (PEEK), polytetrafluoroethylene (PTFE, that is Teflon) or polyethylene terephthalate (PET).

The flexible spine fixing structure 100 may be formed in one piece by way of injection molding or plate bending or others.

Besides, a metal wire may be embedded in the flexible part. For example, in another implementation, if the first flexible part 106 is made from a soft material such as Teflon, then a metal wire (not illustrated) may be embedded in the first flexible part 106 to increase the hardness, the elasticity and the flexibility of the first flexible part 106. If the second flexible part 112 is also made from a soft material, then the same practice may be applied.

The height H1, the interval L1 and the interval W1 may be designed in a manner that the flexible spine fixing structure 100 does not interfere with the vertebra or contact the spinal cord, wherein the height H1 is the distance from the fixing part, such as the second fixing part 110, to the top of the flexible part, the interval L1 is the distance from the through hole of the first fixing part 108 to the through hole of the second fixing part 11, and the interval W1 is the distance from the through hole of the second fixing part 110 to the through hole of the fourth fixing part 116. For example, based on ordinary size of a vertebra, the height H1 may be designed to be between 0 and 25 millimeters (mm), the interval L1 may be designed to be between 30 and 60 mm, and the interval W1 may be designed to be between 10 and 25 mm.

Although the height H1 is exemplified by the distance from the second fixing part 110 to the first flexible part 106, the height H1 of the present embodiment may be the distance from any fixing part to any flexible part.

The height H1 avoids the flexible spine fixing structure 100 interfering with the spinous process of the vertebra or the spinal cord. However, some vertebras are without the spinous process, or the spinous process may be removed in a surgery. Under such circumstances, the height H1 will not interfere with the vertebra or the spinal cord although the height H1 is as low as 0. If the height H1 is 0, this implies that the first flexible part 106, the first fixing part 108 and the second fixing part 110 which are jointed together are substantially straight, and the second flexible part 112, the third fixing part 114 and the fourth fixing part 116 which are jointed together are also straight.

The size ranges of the height H1 and the intervals L1 and W1 disclosed above are not for limiting the present embodiment of the disclosure, and the actual sizes are determined according to the actual size of the surgery recipient's spine. That is, the flexible spine fixing structure 100 of the present embodiment of the disclosure may be tailor-made according to the size of the patient's spine.

### Second Embodiment

Referring to FIG. 3, a schematic view of a flexible spine fixing structure according to a second embodiment of the disclosure is shown. The second embodiment is different from the first embodiment in that: the first flexible part 206 and the second flexible part 212 of the flexible spine fixing structure 200 of the second embodiment are arch-shaped.

The flexible spine fixing structure 200 comprises a first flexible part 206, a first fixing part 208, a second fixing part 210, a second flexible part 212, a third fixing part 214 and a fourth fixing part 216.

The structures of the first fixing part 208, the second fixing part 210, the third fixing part 214 and the fourth fixing part 216 are similar to that of the first fixing part 108, the second fixing part 110, the third fixing part 114 and the fourth fixing part 116 of the first embodiment, and the similarities are not repeated here.

The connection relationships among the first flexible part 206, the first fixing part 208, the second fixing part 210, the second flexible part 212, the third fixing part 214 and the fourth fixing part 216 are similar to that of the first flexible part 106, the first fixing part 108, the second fixing part 110, the second flexible part 112, the third fixing part 114 and the fourth fixing part 116 of the first embodiment, and the similarities are not repeated here.

### Third Embodiment

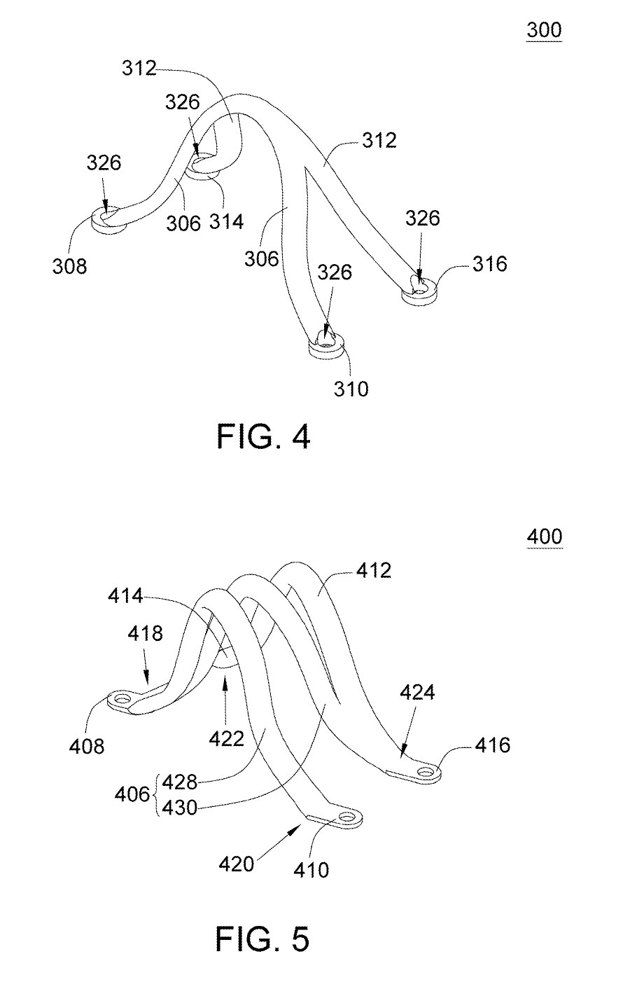

Referring to FIG. 4, a schematic view of a flexible spine fixing structure according to a third embodiment of the disclosure is shown. The third embodiment is different from the second embodiment in that: the fixing part of the flexible spine fixing structure 300 of the third embodiment is a board with through hole.

The flexible spine fixing structure 300 comprises a first flexible part 306, a first fixing part 308, a second fixing part 310, a second flexible part 312, a third fixing part 314 and a fourth fixing part 316.

Any of the first fixing part 308, the second fixing part 310, the third fixing part 314 and the fourth fixing part 316 is a board with through hole 326. The first fixing part 308, the second fixing part 310, the third fixing part 314 and the fourth fixing part 316 may be fixed on the first flexible part 306 and the second flexible part 312 by way of soldering. The through holes 326 allows a number of screws (not illustrated) to pass through and fasten the flexible spine fixing structure 300 on the first vertebra 102 and the second vertebra 104.

The connection relationships among the first flexible part 306, the first fixing part 308, the second fixing part 310, the second flexible part 312, the third fixing part 314 and the fourth fixing part 316 are similar to that the first flexible part 106, the first fixing part 108, the second fixing part 110, the second flexible part 112, the third fixing part 114 and the fourth fixing part 116 of the first embodiment, and the similarities are not repeated here.

### Fourth Embodiment

Referring to FIG. 5, a schematic view of a flexible spine fixing structure according to a fourth embodiment of the disclosure is shown. The third embodiment is different from the first embodiment in that: the first flexible part 406 of the flexible spine fixing structure 400 of the fourth embodiment comprises two sub-flexible parts.

The flexible spine fixing structure 400 comprises a first flexible part 406, a first fixing part 408, a second fixing part 410, a second flexible part 412, a third fixing part 414 and a fourth fixing part 416.

The structures of the first fixing part 408, the second fixing part 410, the third fixing part 414 and the fourth fixing part 416 are similar to that of the first fixing part 108, the second fixing part 110, the third fixing part 114 and the fourth fixing part 116 of the first embodiment, and the similarities are not repeated here.

The first flexible part 406 comprises a first sub-flexible part 428 and a second sub-flexible part 430. The first fixing part 408 and the second fixing part 410 are connected to the first end 418 and the second end 420 of the first sub-flexible part 428 respectively. The third fixing part 414 and the fourth fixing part 416 are connected to the third end 422 and the fourth end 424 of the second flexible part 412 respectively.

The first sub-flexible part 428, the second sub-flexible part 430 and the second flexible part 412 are all arch-shaped. The second sub-flexible part 430 connects one of the first end 418 and the second end 420 of the first sub-flexible part 428 to one of the third end 422 and the fourth end 424 of the second flexible part 412, wherein the two connected ends are the two ends of a diagonal line. For example, the second sub-flexible part 430 connects the first end 418 of the first sub-flexible part 428 to the fourth end 424 of the second flexible part 412. However, the above exemplification is not for limiting the present embodiment of the disclosure. In another implementation, the second sub-flexible part 430 may also connect the second end 420 of the first sub-flexible part 428 to the third end 422 of the second flexible part 412.

The first sub-flexible part 428, the second sub-flexible part 430 and the second flexible part 412 are flexible and the first flexible part 406 is directly connected to the second flexible part 412, so that the entire flexible spine fixing structure 400 is flexible and the fixed vertebra is able to move.

### Fifth Embodiment

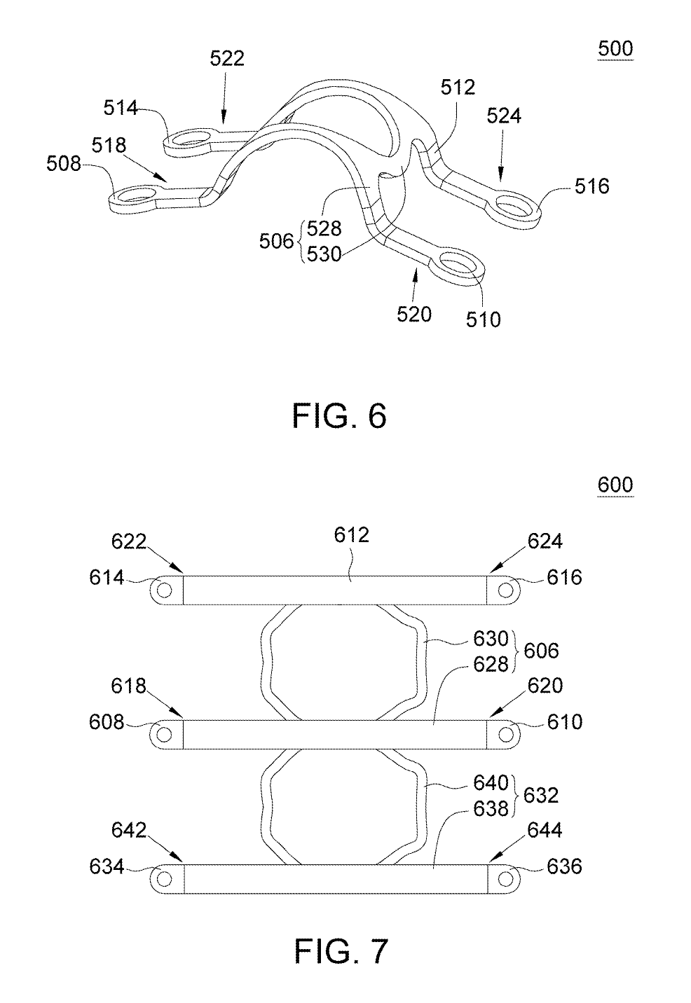

Referring to FIG. 6, a schematic view of a flexible spine fixing structure according to a fifth embodiment of the disclosure is shown. The fifth embodiment is different from the fourth embodiment in that: the second sub-flexible part 530 of the flexible spine fixing structure 500 of the fifth embodiment is directly connected to the arched part of the second flexible part 512 and the arched part of the first sub-flexible part 528.

The flexible spine fixing structure 500 comprises a first flexible part 506, a first fixing part 508, a second fixing part 510, a second flexible part 512, a third fixing part 514 and a fourth fixing part 516.

The structures of the first fixing part 508, the second fixing part 510, the third fixing part 514 and the fourth fixing part 516 are similar to that of the first fixing part 108, the second fixing part 110, the third fixing part 114 and the fourth fixing part 116 of the first embodiment, and the similarities are not repeated here.

The first flexible part 506 comprises a first sub-flexible part 528 and a second sub-flexible part 530. The first fixing part 508 and the second fixing part 510 are connected to the first end 518 and the second end 520 of the first sub-flexible part 528 respectively. The third fixing part 514 and the fourth fixing part 516 are connected to the third end 522 and the fourth end 524 of the second flexible part 512 respectively.

The first sub-flexible part 528 and the second flexible part 512 are all arch-shaped, and the second sub-flexible part 530 is bending, so that the first sub-flexible part 528, the second sub-flexible part 530 and the second flexible part 512 are all flexible. Since the first sub-flexible part 528, the second sub-flexible part 530 and the second flexible part 512 are all flexible and the first flexible part 506 is directly connected to the second flexible part 512, the entire flexible spine fixing structure 500 is thus flexible.

### Sixth Embodiment

Referring to FIG. 7, a schematic view of a flexible spine fixing structure according to a sixth embodiment of the disclosure is shown. The sixth embodiment is different from the fifth embodiment in that: the flexible spine fixing structure 600 of the sixth embodiment further comprises a third flexible part 632, a fifth fixing part 634 and a sixth fixing part 636.

The flexible spine fixing structure 600 comprises a first flexible part 606, a first fixing part 608, a second fixing part 610, a second flexible part 612, a third fixing part 614 and a fourth fixing part 616. The first fixing part 608 and the second fixing part 610 are connected to the first end 618 and the second end 620 of the first flexible part 606 respectively. The third fixing part 614 and the fourth fixing part 616 are connected to the third end 622 and the fourth end 624 of the second flexible part 612 respectively.

The first flexible part 606 comprises a first sub-flexible part 628 and a second sub-flexible part 630. The first fixing part 608 and the second fixing part 610 are connected to the first end 618 and the second end 620 of the first sub-flexible part 628 respectively. The third fixing part 614 and the fourth fixing part 616 are connected to the third end 622 and the fourth end 624 of the second flexible part 612 respectively.

The third flexible part 632 comprises a third sub-flexible part 638 and a fourth sub-flexible part 640. The fifth fixing part 634 and the sixth fixing part 636 are connected to a fifth end 642 and a sixth end 644 of the third sub-flexible part 638 respectively for fixing in a third vertebra (not illustrated).

The fourth sub-flexible part 640 is directly connected to the first sub-flexible part 628. Thus, the flexible spine fixing structure 600 forms three groups of fixing parts for fixing in three adjacent vertebras, such as the first vertebra, the second vertebra and the third vertebra respectively.

In the present embodiment of the disclosure, the number of groups of fixing parts is exemplified by three. However, in other implementations, the fixing parts may have more than three groups.

### Seventh Embodiment

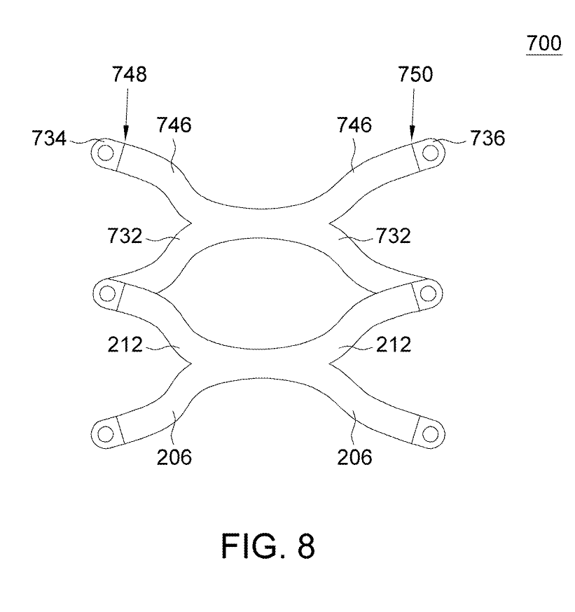

Referring to FIG. 8, a schematic view of a flexible spine fixing structure according to a seventh embodiment of the disclosure is shown. As for the similarities between the seventh embodiment and the second embodiment, the same designations are used, and the similarities are not repeated here. The seventh embodiment is different from the second embodiment in that: the flexible spine fixing structure 700 of the seventh embodiment further comprises a third flexible part 732, a fourth flexible part 746, a fifth fixing part 734 and a sixth fixing part 736.

The fifth fixing part 734 and the sixth fixing part 736 are connected to a seventh end 748 and an eighth end 750 of the fourth flexible part 746 respectively for fixing in a third vertebra (not illustrated). The two ends of the third flexible part 732 are connected to the second flexible part 212. Thus, the flexible spine fixing structure 700 form three groups of fixing parts for fixing in three adjacent vertebras, such as the first vertebra, the second vertebra and the third vertebra respectively.

In the present embodiment of the disclosure, the number of groups of fixing parts is exemplified by three. However, in other implementations, the fixing parts may have more than three groups.

Anyone who is skilled in the technology of the disclosure will understand that the fixing part of the first, the second, and the fourth to the seventh embodiment may be replaced by board with through hole as disclosed in the third embodiment. The technical features of the board with through hole are already disclosed in the third embodiment, and are not repeated here.

If the material of the flexible part of the second embodiment to the seventh embodiment is soft, a metal wire may be disposed in the flexible part to increase the hardness, elasticity and flexibility of the flexible part as disclosed in the first embodiment. The technical features of the metal wire are already disclosed in the first embodiment, and are not repeated here.

Besides, the size relationships between the flexible part and the fixing part of the second embodiment to the seventh embodiment are similar to that of the first embodiment, and the similarities are not repeated here.

According to the flexible spine fixing structure disclosed in the above embodiments of the disclosure, the second flexible part is directly connected to the first flexible part, so that the entire flexible spine fixing structure is flexible. After the flexible spine fixing structure is fixed on the vertebra, the vertebra being fixed is still moveable and allows the patient to move forward, backward, leftward or rightward, and provides the patient with higher degree of activities, so that so that the patient's discomfort after surgery is reduced, and spine degeneration will be avoided.

While the disclosure has been described by way of example and in terms of a preferred embodiment, it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A flexible spine fixing structure for fixing in a first vertebra and a second vertebra of a spine, wherein the flexible spine fixing structure comprises:
a first flexible part;
a first fixing part and a second fixing part connected to two ends of the first flexible part respectively for fixing in the first vertebra;
a second flexible part directly connected to the first flexible part;
a third fixing part and a fourth fixing part connected to two ends of the second flexible part respectively for fixing in the second vertebra.

2. The flexible spine fixing structure according to claim 1, wherein the first flexible part, the first fixing part, the second fixing part, the second flexible part, the third fixing part and the fourth fixing part are integrally formed in one piece.

3. The flexible spine fixing structure according to claim 1, wherein at least one of the first flexible part and the second flexible part is arch-shaped.

4. The flexible spine fixing structure according to claim 3, wherein the height between the arched top of the first flexible part and at least one of the first fixing part and the second fixing part ranges between 0 to 25 millimeters (mm), and the height between the arched top of the second flexible part and at least one of the third fixing part and the fourth fixing part ranges between 0 to 25 mm.

5. The flexible spine fixing structure according to claim 1, wherein the angle contained between the first fixing part and the first flexible part, the angle contained between the second fixing part and the first flexible part, the angle contained between the third fixing part and the second flexible part and the angle contained between the fourth fixing part and the second flexible part are obtuse angles.

6. The flexible spine fixing structure according to claim 1, wherein the angle contained between the first fixing part and the first flexible part, the angle contained between the second fixing part and the first flexible part, the angle contained between the third fixing part and the second flexible part and the angle contained between the fourth fixing part and the second flexible part ranges between 120-150 degrees.

7. The flexible spine fixing structure according to claim 1, wherein the angle contained between the first fixing part and the first flexible part, the angle contained between the second fixing part and the first flexible part, the angle contained between the third fixing part and the second flexible part, and the angle contained between the fourth fixing part and the second flexible part are substantially 180 degrees.

8. The flexible spine fixing structure according to claim 1, wherein the first flexible part comprises a first sub-flexible part and a second sub-flexible part, the first fixing part and the second fixing part are connected to the two ends of the first sub-flexible part respectively, and the second sub-flexible part connects the first sub-flexible part and the second flexible part.

9. The flexible spine fixing structure according to claim 8, wherein the second sub-flexible part connects one of the two ends of the first sub-flexible part to one of the two ends of the second flexible part, and the two connected ends are two ends of a diagonal line.

10. The flexible spine fixing structure according to claim 8, further comprising:
a third flexible part; and
a fifth fixing part and a sixth fixing part connected to two ends of the third flexible part respectively for fixing in a third vertebra;
wherein, the third flexible part comprises a third sub-flexible part and a fourth sub-flexible part, the fifth fixing part and the sixth fixing part are connected to two ends of the third sub-flexible part respectively, and the fourth sub-flexible part is connected to the second sub-flexible part.

11. The flexible spine fixing structure according to claim 1, further comprising:
a third flexible part;
a fourth flexible part; and
a fifth fixing part and a sixth fixing part connected to two ends of the fourth flexible part respectively for fixing in a third vertebra;
wherein, the two ends of the third flexible part is connected to one of the first flexible part and the second flexible part.

12. The flexible spine fixing structure according to claim 1, wherein any of the first fixing part, the second fixing part, the third fixing part and the fourth fixing part has a through hole.

13. The flexible spine fixing structure according to claim 1, wherein the first flexible part and the second flexible part are integrally formed in one piece, and any of the first fixing part, the second fixing part, the third fixing part and the fourth fixing part respectively is a board with through hole.

14. The flexible spine fixing structure according to claim 1, wherein any of cross-sections of the first flexible part and the second flexible part is rectangular, circular or elliptical.

15. The flexible spine fixing structure according to claim 1, wherein the first flexible part and the second flexible part are made from metal.

16. The flexible spine fixing structure according to claim 15, wherein the metal is realized by stainless steel, memory alloy, or metal at least containing titanium.

17. The flexible spine fixing structure according to claim 1, wherein the first flexible part and the second flexible part are made from polymer.

18. The flexible spine fixing structure according to claim 1, wherein the first flexible part and the second flexible part are made from polytetrafluoroethylene (Teflon).

19. The flexible spine fixing structure according to claim 1, further comprising:
a metal wire embedded in one of the first flexible part and the second flexible part.
